(19) United States
(12) Patent Application Publication
(10) Pub. No.: US 2008/0308748 A1
(43) Pub. Date: Dec. 18, 2008

(54) Apparatus and method of disinfecting footwear

(76) Inventor: Christo Nico William Burrows, Toronto (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 11/808,928

(22) Filed: Jun. 13, 2007

Related U.S. Application Data
(60) Provisional application No. 60/804,596.

(51) Int. Cl. A61L 2/10

(52) U.S. Cl. 250/491.1

(57) ABSTRACT

A footwear sterilization and disinfection apparatus and method using UV-C light to sterilize or disinfect the inside of the footwear is provided. The source of the UV-C light may be a singular or a plurality of sources and may include a solid state source, mercury vapour bulb or one or more light emitting diodes. The apparatus for sterilizing and disinfecting footwear may either be a separate device which may be positioned within the footwear or can be provided as part of the footwear during construction regarding manufacture of the footwear. For safety purposes, a sealed housing or shrouding curtain may be provided so that the radiation of UV-C light takes place within the sealed housing or behind the shrouding curtain so that a user is protected from radiation by UV-C light.

### RELATED APPLICATION

This application claims the benefit under 35 U.S.C. 119(e) to U.S. Provisional Application No. 60/804,596, filed Jun. 13, 2006.

### FIELD OF THE INVENTION

This invention relates to an apparatus and method of disinfecting footwear by emitting ultra violet light in an interior of the footwear. More specifically, the invention relates to an apparatus having a positioning member that fits entirely within footwear and holds a source of ultra violet light to disinfect or sanitize the footwear.

### BACKGROUND OF THE INVENTION

It is known to spray chemicals into footwear to disinfect or sterilize the inside of the footwear. However, spraying the chemicals does not accurately ensure that the disinfectant is applied to an entirety of the interior of the shoe. In particular, it is difficult to spray the chemical into the toe portion given the dynamics of inserting a spray bottle at a heel portion of the footwear and spraying towards the toe portion of the footwear. This is further complicated with the boots which have a tall upper part of the heel. Also, spraying chemicals is messy and unreliable in terms of disinfecting and sanitizing. It is also known to apply medicament or cream to a person's foot to treat bacterial or fungus growth such as athlete's foot. However, this treatment does not address the source of the bacteria or fungus and does not treat the bacteria or fungal growth in the footwear.

It is also known to use ultra violet light sources to sterilize or disinfect instruments in the healthcare industry, including endoscopes. U.S. Pat. No. 7,175,806 issued Feb. 13, 2007, discloses a UV-C band disinfector device which disinfects objects such as endoscopes which are placed within the device by subjecting the objects to emissions of UV-C radiation. This patent also teaches use of fibre-optics that transmit UV-C radiation to disinfect interior chambers, channels and conduits of the endoscopes. A metal housing is provided with medium pressure mercury bulbs that produce ultra violet light in the UV-C range housed in a vertical chamber to radiate the interior or the exterior of an object such as a medical instrument.

U.S. Pat. No. 7,217,936 issued May 15, 2007, teaches the use of UV light to sterilize polymer based products such as packaging for meat and poultry products or contact lenses. This patent teaches the use of conveyor belts or tracks to transport products through an intermediate region for UV radiation and sterilization treatment.

However, there remains a need for an apparatus and method of disinfecting the interior of footwear and in particular a need to provide an apparatus which fits entirely within the footwear to provide sanitation and disinfecting thereto. While a ultra violet light wand is known for use in household disinfecting applications to disinfect viruses, bacteria, mold and mildew, this wand is not suitable for use in disinfecting the interior of footwear as the wand can not fit entirely within the footwear. Also, the wand exposes the user to ultra violet light which is harmful and thus presents a safety hazard.

The applicant has therefore appreciated that there is a need for a device and method of disinfecting or sterilizing an interior of footwear.

### SUMMARY OF THE INVENTION

The present invention is directed to an apparatus and method of disinfecting footwear in particular the interior of the footwear using ultra violet-C (UV-C) radiation. The disclosed apparatus and method of disinfecting footwear is effective in sterilizing or disinfecting viral and bacterial micro organisms without damaging the footwear or posing a health risk to a person using the apparatus or carrying our the method.

By providing a positioning member sized to be fitted within footwear and adapted to receive the source of ultra violet light, the apparatus for disinfecting footwear can be placed entirely within the footwear and the ultra violet light can be radiated therein to disinfect or sanitize the footwear. The positioning member can comprise a semi-cylindrical dome shape positionable with longitudinal edges of the dome-shaped member against the sole of the footwear and with the ultra violet light source positioned adjacent a concave side of the semi-cylindrical dome shape above a sole of the footwear. This construction enables the positioning of the source of ultra violet light entirely within the interior of the footwear and without directly touching the footwear.

Alternatively, the positioning member may be an insert or insole having a contour sized to be positionable inside the footwear adjacent a sole of the footwear. The source of ultra violet light could be one or more light emitting diodes which are surface mounted to the insert. In another embodiment, the insole could be formed of a fibre optic weave used to distribute light throughout the interior of the footwear. In an alternative embodiment, the source of ultra violet light is provided in the sole of the shoe during manufacture. The source of ultra violet light could be connected with the fibre optic weave to distribute the ultra violet light.

As an added safety feature, a sealed housing or shrouding could also be provided so that the disinfecting or sterilizing of the footwear can only be performed when the footwear with the apparatus positioned within the housing or behind the shrouding curtain to protect a user from being radiated with the UV light.

In one aspect, the present invention resides in an apparatus for disinfecting footwear comprising: a positioning member sized to be fitted within the footwear and adapted to receive a source of ultra violet light to disinfect the footwear.

Further and other features of the invention will be apparent to those skilled in the art from the following detailed description of the embodiments thereof.

### BRIEF DESCRIPTION OF THE DRAWINGS

Reference may now be had to the following detailed description taken together with the accompanying drawings in which:

FIG. 1 shows a shoe having a disinfecting and sterilizing apparatus positioned therein;

FIG. 2 shows the disinfecting and sterilizing apparatus shown in FIG. 1;

FIG. 3 shows the disinfecting and sterilizing apparatus positioned within the footwear as shown in FIG. 1 from a side view;

FIG. 4 shows another embodiment of the disinfecting and sterilizing apparatus having light emitting diodes in an insert which is positionable within a shoe; and

FIG. 5 shows another embodiment of the disinfecting and sterilizing apparatus having an insert with a fibre optic weave embedded therein which is positionable within a shoe.

### DETAILED DESCRIPTION OF THE PREFERRED EMBODIMENTS

FIG. 1 shows a shoe 2 having an interior sole 4 on which a disinfecting sterilizing apparatus 6 is positioned. The disinfecting and sterilizing apparatus 6 is a separate device which is sized to be positioned entirely within the interior of the shoe 2 and to radiate ultra violet light and disinfect and sterilize the interior of the shoe 2.

The apparatus 6 has a toe positioning member 8 and a heel positioning member 10 connected by a conduit 12. The toe positioning member 8 and heel positioning member 10 have a substantially semi-cylindrical shaped contour with longitudinal edges positioned against the sole 4 of the shoe 2. In a preferred embodiment, the apparatus 6 may include only the toe positioning member 8 or heel positioning member 10 and would not need to be connected with a conduit 12. Also, the toe positioning member 8 and/or heel positioning member 10 can be opaque with a reflective surface on an underside thereof to reflect ultra violet light downwardly onto the sole 4 of the shoe 2 to contain the ultra violet light within an interior of a shoe 2. Alternatively, the toe positioning member 8 and heel positioning member 10 can be transparent or translucent and allow the ultra violet light to radiate therethrough.

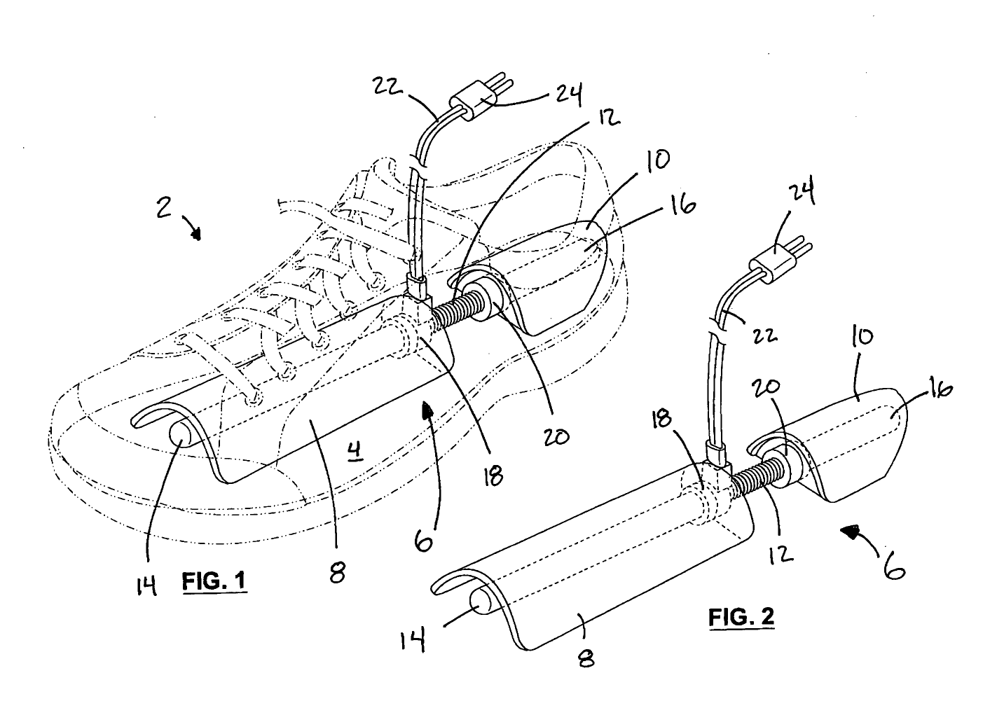

As also shown in FIG. 2, the ultra violet light is provided by a toe ultra violet-C (UV-C) bulb 14 and heel UV-C bulb 16 fitted within toe receptacle 18 and heel receptacle 20, respectively. The toe UV-C bulb 14 and heel UV-C bulb 16 are positioned within the receptacles and adjacent to the toe positioning member 8 and heel positioning member 10 respectively so that the bulb is maintained within the interior of the shoe 2 does not contact the interior of the shoe. For example, with respect to the toe positioning member 8, the longitudinal edges are positioned against the sole 4 to hold the toe UV-C bulb 14 in a suspended state so that it does not contact the sole 4 of the shoe 2. The heel UV-C bulb 16 is supported in a similar fashion by the heel positioning member 10.

The toe UV-C bulb 14 and heel UV-C bulb 16 can be mercury vapour bulbs which emit UV-C light having a wavelength of 280 nm or less. Preferably, the wavelength of the UV-C light is between 180 and 280 nm. More preferably, a wavelength of 185 nm or 265 nm is radiated as these wavelengths advantageously achieve better disinfection and sterilization of bacteria and viruses.

The toe UV-C bulb 14 and heel UV-C bulb 16 are provided with power via the cord 22 electrically connected at first end to the toe receptacle 18 and having a plug 24 connected to a second end of the cord 22. The plug 24 can be inserted to a jack (not shown) which provides electricity to power the toe UV-C bulb 14 and heel UV-C bulb 16.

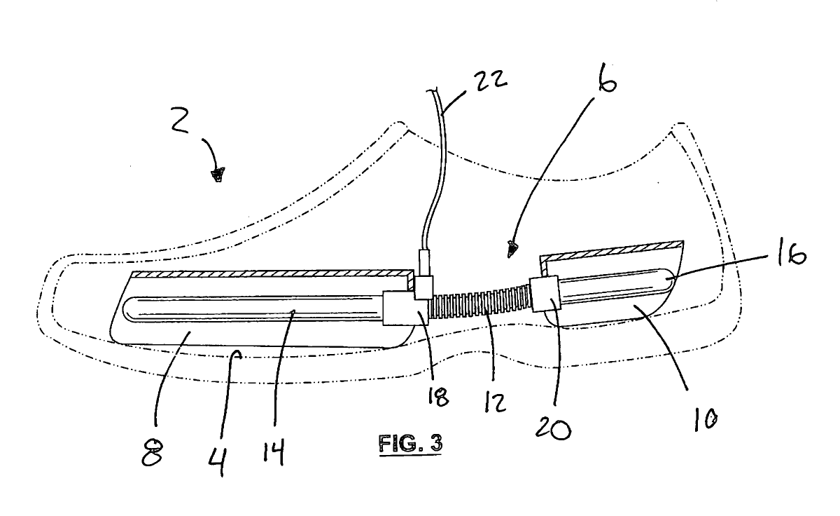

FIG. 3 shows the apparatus 6 fitted within the shoe 2 from a side view. In a preferred embodiment, the conduit 12 has threads 26 which meet with corresponding threads in the toe receptacle 18 and heel receptacle 20, so that the apparatus 6 can be lengthened or shortened.

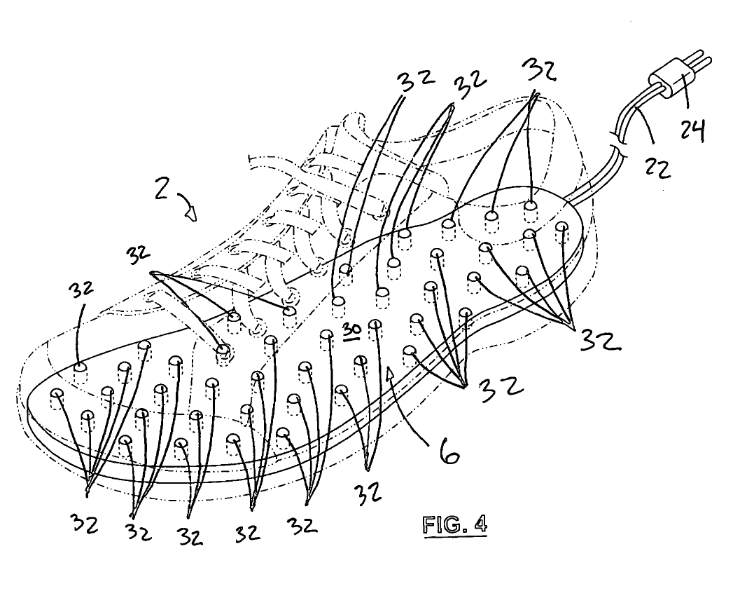

FIG. 4 shows another embodiment of the present invention wherein the positioning member is an insert or insole 30 which is contoured to fit within the interior of the shoe 2 on top of the sole. The insole 30 has a plurality of light emitting diodes (LEDs) 32 which are surface mounted in the insole. The plurality of LEDs 32 are electrically connected to one another and to the power cord 22. The plug 24 is connected to the power cord for fitting into a receptacle to provide power to the plurality of LEDs 32.

The LEDs emit UV-C light to the interior of the shoe to disinfect and sterilize the interior of the shoe 2. Preferably, the wavelength of the UV-C light is between 180 and 200 nm. In a preferred embodiment, the LEDs 32 can be tuned to emit UV-C light having wavelength of either 185 nm or 265 nm. The applicant has appreciated that these wavelengths provide optical disinfecting and sterilizing of bacteria and viruses.

In an alternative embodiment, the insole 30 could be permanently fixed within the shoe 2. In this embodiment, the power source receptacle (not shown) could be provided on the shoe for connection with a power source.

As a further safety feature to ensure that a user does not come in contact with the UV-C radiation, a sealed housing or shrouding curtain (not shown) can be provided so that the radiation takes place within the sealed housing or shrouding curtain. An example of the sealed housing could be a metal box which the shoe having the sterilizing and disinfecting apparatus positioned therein can be placed and closed during the period of time when the UV radiation is applied within the shoe.

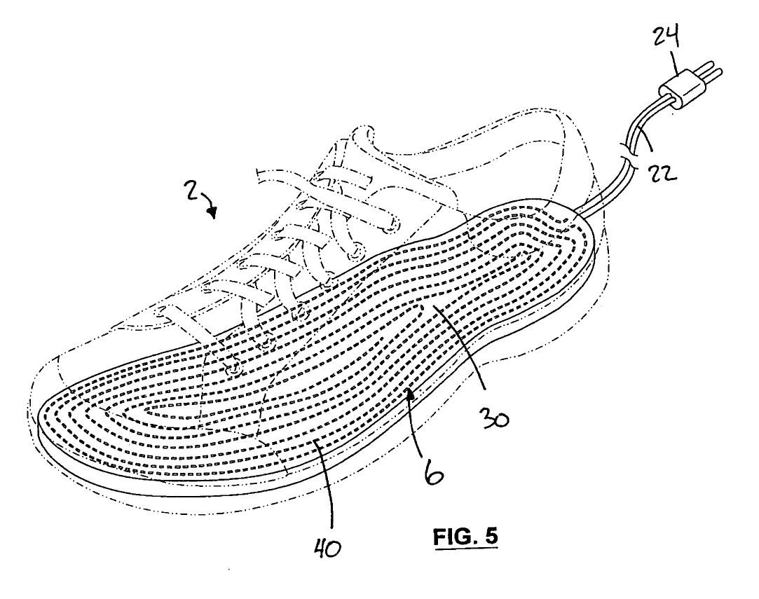

As shown in FIG. 5, the insole 30 comprises a fibre optic weave 40 embedded therein which is used to distribute light throughout the footwear or shoe 2. For example, the source of the UV-C light could be a single or a plurality of mercury vapour bulbs (not shown) contained within a fixture that connects to a receptacle in either the heel or the toe of the footwear and projects the UV-C light into the fibre optic weave to distribute the light to the remainder of the footwear. Alternatively, the fibre optic weave 40 could be formed as part of the shoe 2 during manufacture thereof.

Although this disclosure has described and illustrated certain preferred embodiments of the invention, it is also to be understood that the invention is not restricted to these particular embodiments rather, the invention includes all embodiments which are functional, or mechanical equivalents of the specific embodiments and features that have been described and illustrated herein.

It will be understood that, although various features of the invention have been described with respect to one or another of the embodiments of the invention, the various features and embodiments of the invention may be combined or used in conjunction with other features and embodiments of the invention as described and illustrated herein.

The embodiments of the invention in which an exclusive property or privilege is claimed is defined as follows:

1. An apparatus for disinfecting footwear comprising:
a positioning member sized to be fitted within the footwear and adapted to receive a source of ultra violet light to disinfect the footwear.

2. The apparatus of claim 1, wherein the positioning member has an elongated semi-cylindrical dome shape so as to be positionable with longitudinal edges of the positioning member the against a sole of the footwear and source of ultra violet light positioned adjacent a concave side of the semi-cylindrical dome shaped positioning member to suspend the source of ultra violet light above the sole of the footwear and to prevent the source of ultra violet light from directly touching the footwear.

3. The apparatus of claim 2, wherein the source of ultra violet light is a bulb.

4. The apparatus of claim 3, wherein the ultra violet light has a wavelength of between 180 nm and 280 nm.

5. The apparatus of claim 4, wherein the ultra violet light has a wavelength of 254 nm.

6. The apparatus of claim 4 wherein the ultra violet light has a wavelength of 185 nm.

7. The apparatus of claim 4 wherein the ultra violet light has a wavelength of 265 nm.

8. The apparatus of claim 1, wherein the source of ultra violet light is a mercury vapour bulb.

9. The apparatus of claim 1, wherein the source of ultra violet light is at least one light emitting diode (LED).

10. The apparatus of claim 1, wherein the positioning member is an insert having a contour sized to be positionable inside the footwear adjacent sole of the footwear.

11. The apparatus of claim 10, wherein at least one light emitting diode (LED) is surface mounted to the insert.

12. The apparatus of claim 11, wherein a plurality of light emitting diodes (LED) are surface mounted to heel and toe portions of the insert.

13. The apparatus of claim 1, wherein the positioning member is an insert having a contour sized to be positionable inside the footwear adjacent sole of the footwear, and the insert comprises fibre optic weave.
